(19) United States
(12) Patent Application Publication
(10) Pub. No.: US 2017/0368031 A1
(43) Pub. Date: Dec. 28, 2017

(54) SOLID DISPERSION FORMULATIONS OF ANTIVIRAL COMPOUNDS

(71) Applicants: Agam SHETH, West Point, PA (US); Pavithra SUNDARARAJAN, West Point, PA (US); Elise MILLER, West Point, PA (US); Justin David MOSER, West Point, PA (US); Adam SOCIA, West Point, PA (US); Angela M. WAGNER, West Point, PA (US); Merck Sharp & Dohme Corp., Rahway, NJ (US)

(72) Inventors: Agam SHETH, Plymouth Meeting, PA (US); Pavithra SUNDARARAJAN, North Wales, PA (US); Elise MILLER, Gilbertsville, PA (US); Justin David MOSER, Collegeville, PA (US); Adam J SOCIA, Blue Bell, PA (US); Angela M WAGNER, Austin, TX (US)

(73) Assignee: Merck Sharp & Dohme Corp., Rahway, NJ (US)

(21) Appl. No.: 15/537,564

(22) PCT Filed: Nov. 6, 2015
(86) PCT No.: PCT/US15/59372

§ 371 Date: Jun. 19, 2017

Related U.S. Application Data
(60) Provisional application No. 62/095,398, filed on Dec. 22, 2014.

(51) Int. Cl. A61K 31/4155; A61K 45/06; A61K 9/16; A61K 31/421

(52) U.S. Cl. CPC A61K 31/4155; A61K 31/421; A61K 45/06; A61K 9/1617; A61K 9/1652; A61K 2300/00

(57) ABSTRACT

The present disclosure is directed to solid dispersion formulations comprising an NS5A inhibitor compound, elbasvir (dimethyl N,N′-([(6S)-6-phenylindolo [1,2- c][1,3]benzoxazine-3,10-diyl]bis {1H-imidazole-5,2-diyl-(2S)-pyrrolidine-2,1-diyl[(2S)-3-methyl-l-oxobutane-1,2-diyl]})di-carbamate), or a pharmaceutically acceptable salt thereof, a pharmaceutically acceptable polymer, and optionally a pharmaceutically acceptable surfactant. The present disclosure is also directed to solid dispersion formulations, blended compositions and pharmaceutical dosage forms containing or made from these solid dispersion formulations, and the methods for making these solid dispersion formulations, blended compositions and pharmaceutical dosage forms.

### FIELD OF THE INVENTION

The instant invention relates to pharmaceutical formulations that may be useful for the treatment of diseases and disorders caused by hepatitis C virus (“HCV”). In particular, the pharmaceutical formulations include solid dispersion formulations that comprise an antiviral compound.

### BACKGROUND OF THE INVENTION

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-a alone or in combination with the nucleoside analog ribavirin. Potential treatments for HCV infection have been discussed in the different references including Balsano, 8(4) MINI REV. MED. CHEM. 307-318, 2008, Rönn et al., 8 CURRENT TOPICS IN MEDICINAL CHEMISTRY 533-562, 2008, Sheldon et al., 16(8) EXPERT OPIN. INVESTIG. DRUGS 1171-1181, 2007, and De Francesco et al., 58 ANTIVIRAL RESEARCH 1-16, 2003. Several virally-encoded enzymes are putative targets for therapeutic intervention, including a metalloprotease (NS2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B).

One identified target for therapeutic intervention is the HCV NS5A non-structural protein, which is described, for example, in Seng-Lai Tan & Michael G. Katze, 284 VIROLOGY 1-12 (2001); and in Kyu-Jin Park et al., 278(33) J. BIO. CHEM. 30711 (2003). A non-structural protein, NS5A is an essential component for viral replication and assembly. Mutations in NS5A at or near known sites of phosphorylation can affect the ability for high-level replication in cell-culture systems, suggesting an important role for NS5A phosphorylation in viral replication efficiency. Inhibitors of the phosphorylation of NS5A can lead to reduced viral RNA replication. NS5A inhibitor compounds include compounds such as elbasvir (dimethyl N,N′-([(6S)-6-phenylindolo[1,2-c][1,3]benzoxazine-3,10-diyl]bis {1H-imidazole-5,2-diyl-(2S)-pyrrolidine-2,1-diyl[(2S)-3-methyl-l-oxobutane-1,2-diyl]})dicarbamate), which is shown below as Compound I:

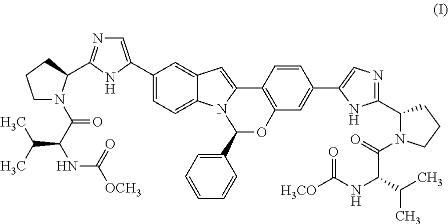 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]

Compound I is described in U.S. Pat. No. 8,871,759.

Compound I, a weak base, has two basic sites, which protonate at low pH giving rise to a sharp pH-solubility profile, particularly between pH 1-3; a normal human stomach has a pH in a range from 1-3 but usually closer to 2. The steep profile has practical implications for dissolution and absorption of Compound I, and likewise for the dissolution and absorption of other weak bases, in the gastrointestinal tract of patients. Specifically, the amount of drug dissolved from formulations of weakly basic compounds in patients with elevated gastric pH could be significantly impaired and more variable, which in turn could lead to potentially lower absorption. See E. Lahner et al., 29 ALIMENTARY PHARMACOL. THER. 1219-1229 (2009); T. L. Russell et al., 11(1) PHARM. RES. 136-143 (1994); G. Krishna et al., 53(3) ANTIMICROB. AGENTS CHEMOTHER. 958-966 (2009).

Elevated gastric pH, or reduced gastric acidity, is known as achlorhydria and can result from a variety of factors. See A. Mitra & F. Kesisoglou, 10 MOL. PHARM. 2970-2979 (2013). Absorption of several drugs such as ketoconazole, itraconazole, atazanavir, cefpodoxime, enoxacin, dipyridamole, nifedipine and digoxin has been shown to be impaired due to this condition. See E. Lahner et al., 29 ALIMENTARY PHARMACOL. THER. 1219-1229 (2009).

Because of the importance of gastric pH in driving dissolution, absorption, and ultimately efficacy of weakly basic compounds such as Compound I, it is imperative to develop formulations that can minimize or mitigate the effects of increased gastric pH on Compound I′s bioavailability. Such formulations may prove particularly useful in the treatment of HIV patients who are coinfected with HCV. About one-quarter of HIV-infected persons in the United States are also infected with HCV, and these patients tend to have higher gastric pH. See HIV and

Viral Hepatitis Fact Sheet, Centers for Disease Control and Prevention (March 2014), available online at http://www.cde.gov/hepatitis/Populations/PDFsiFiniandliep-FactSheet.pdf.

Solid dispersion formulations have been used previously to minimize the effect of achlorhydria for weak bases. See M.A. Alam et al., 9(11) EXPERT OPIN. DRUG DELIVERY 1419-1440 (2012); A. Mitra et al., 8 Mol. Pharm. 2216-2223 (2011). Solid dispersion formulations are compositions in which one or more active pharmaceutical ingredient (API) is dispersed into excipients. Solid solutions, defined as solid dispersions in which the API forms a homogeneous or nearly homogeneous glass when dispersed into the excipient matrix, are of particular interest in the oral delivery of compounds that are sensitive to gastric pH. Solid dispersion formulations as described above may provide enhanced insensitivity to variations in gastric pH relative to crystalline forms of the API. There remains a need for formulations that provide enhanced insensitivity to variations in gastric pH relative to other formulations containing amorphous forms of the API.

In addition, despite their growing use, the design of solid solution formulations to effectively promote oral drug absorption remains largely a matter of trial and error. Thus, there also remains a need for solid dispersion formulations of drug substances that may provide effective absorption following oral administration, which is useful to reduce pill burden (e.g., the number of tablets administered), regimen complexity (e.g., eliminating the need to administer with food), and facilitate co-dosing with other medications, such as antacid medications. Formulations with this type of enhanced absorption will ultimately improve compliance, and, therefore, efficacy.

The current invention relates to novel formulations based on solid dispersion formulations, which may confer insensitivity to higher gastric pH by one or more of the following: enhancing dissolution rate, modulating particle sizes and/or maintaining higher supersaturation of Compound I relative to its amorphous form.

### SUMMARY OF THE INVENTION

The present disclosure relates to elbasvir (dimethyl N,N′-([(65)-6-phenylindolo[1,2-c][1,3]benzoxazine-3,10-diylibis {1H-imidazole-5,2-diyl-(2S)-pyrrolidine-2,1-diyl[(2S)-3-methyl-l-oxobutane-1,2-diyl]})dicarbamate), shown below as Compound I:

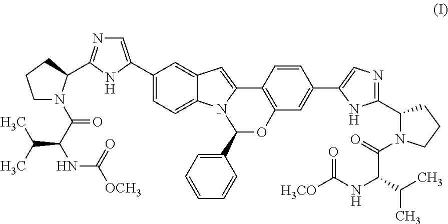 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]

or a pharmaceutically acceptable salt thereof, formulated into solid dispersion formulations and pharmaceutical dosage forms. In embodiments, solid dispersion formulations of the disclosure may provide insensitivity to gastric pH. Insensitivity to increases in gastric pH potentially may be provided by mechanisms such as increased dissolution rates, modulation of particle sizes and/or maintenance of supersaturation.

Other embodiments, aspects and features of the present invention are either further described in or will be apparent from the ensuing description, examples and appended claims.

### BRIEF DESCRIPTION OF THE DRAWINGS

FIG. 1 provides a schematic representation of the formulation process for preparing Formulation 1.

FIG. 2 provides a schematic representation of the formulation process for preparing Formulation 2.

FIG. 3 provides a schematic representation of the formulation process for preparing Formulation 3.

### DETAILED DESCRIPTION OF THE INVENTION

The instant disclosure is directed to a solid dispersion formulation comprising (a) Compound I or a pharmaceutically acceptable salt thereof; (b) one or more pharmaceutically acceptable polymers; and (c) optionally one or more pharmaceutically acceptable surfactants. The disclosure is also directed to blended compositions comprising such solid dispersion formulations, and oral dosage forms, such as tablets or capsules comprising such solid dispersion formulations and/or blended compositions comprising such solid dispersion formulations.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations off ±1%, ±2%, ±3%, ±4%, ±5%, ±10%, ±15%, and ±20% and their numerical equivalents.

As used herein, the term “one or more” item includes a single item selected from the list as well as mixtures of two or more items selected from the list.

As used herein, the term “amorphous” indicates that the material lacks order on a molecular level and may exhibit the physical properties of a solid or a liquid, depending on the temperature of the material. Amorphous materials do not give distinctive X-ray diffraction patterns.

As used herein, the term “crystalline” indicates that the material has a regular ordered internal structure at the molecular level in the solid form, and the crystalline material gives a distinctive X-ray diffraction pattern with defined peaks.

As used herein, the term “substantially amorphous” refers to a composition in which greater than about 70%; or greater than about 75%; or greater than about 80%; or greater than about 85%; or greater than about 90%; or greater than about 95%, or greater than about 99% of the compound present is amorphous. “Substantially amorphous” can also refer to material that has no more than about 20% crystallinity, or no more than about 10% crystallinity, or no more than about 5% crystallinity, or no more than about 2% crystallinity.

The term “effective amount” indicates a sufficient amount to exert a therapeutic or prophylactic effect. For a patient who is infected with HCV, an effective amount is sufficient to achieve one or more of the following effects: reduce the ability of HCV to replicate, reduce HCV load, and increase viral clearance. For a patient who is not infected with HCV, an effective amount is sufficient to achieve one or more of the following: a reduced susceptibility to HCV infection, and a reduced ability of the infecting virus to establish persistent infection for chronic disease.

The term “subject” (alternatively referred to herein as “patient”) as used herein refers to an animal, preferably a mammal, most preferably a human, who has been the object of treatment, observation or experiment.

The term “pharmaceutically acceptable salt” refers to a salt of the parent compound that has activity and that is not biologically or otherwise undesirable (e.g., is neither toxic nor otherwise deleterious to the recipient thereof); also included in this term are complexes that comprise solvent molecules and a salt of the parent compound. Suitable salts include acid addition salts that may, for example, be formed by mixing a solution of a compound with a solution of a pharmaceutically acceptable acid such as hydrochloric acid, sulfuric acid, trifluoroacetic acid, phosphoric acid, methanesulfonic acid, naphthalene-1,5-disulfonic acid or toluenesulfonic acid. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts), and salts formed with suitable organic ligands, such as quaternary ammonium salts. Also, in the case of an acid (-COOH) or alcohol group being present, pharmaceutically acceptable esters can be employed to modify the solubility or hydrolysis characteristics of the compound.

The term “polymer” as used herein refers to a chemical compound or mixture of compounds consisting of repeating structural units created through a process of polymerization. Suitable polymers useful in this invention are described throughout. When specific polymers that are suitable for use in the compositions of the present invention are blended, the blends of such polymers may also be suitable. Thus, the term “polymer” is intended to include blends of polymers in addition to a single species of polymer.

In the embodiments described herein, any variable or component is as defined in the first instance where the variable or component occurs, unless otherwise indicated. When any variable or component occurs more than one time, its selection on each occurrence is independent of its selection at every other occurrence. Also, combinations of embodiments, variables or components are permissible only if such combinations result in stable formulations, blends, or oral dosage forms.

### Solid Dispersion Formulations

In a first embodiment, the instant disclosure is directed to a solid dispersion formulation comprising (a) Compound I or a pharmaceutically acceptable salt thereof; (b) one or more pharmaceutically acceptable polymers; and (c) optionally one or more pharmaceutically acceptable surfactants. Compound I may be amorphous or substantially amorphous.

In a first aspect of the first embodiment, Compound I, or a pharmaceutically acceptable salt thereof, is present in a concentration of from about 5% w/w to about 50% w/w, based on the total combined weight of the drug substance, polymer, and any optional surfactant (not including other excipients). In particular instances, Compound I, or a pharmaceutically acceptable salt thereof, is present in a concentration of from about 10% w/w to about 40% w/w, or about 20% w/w, based on the total combined weight of the drug substance, polymer, and any optional surfactant (not including other excipients). In this first embodiment, all other variables are as provided above.

In a second aspect of the first embodiment, the one or more pharmaceutically acceptable polymer is non-ionic.

The one or more pharmaceutically acceptable polymers are selected from the group consisting of cellulosic polymers and vinyl pyrrolidinone/vinyl acetate copolymers.

Cellulosic polymers include cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate); cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate (HPMCA5)). Commercially available examples of these include hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, and HPMC P 55. In particular aspects of this embodiment, the one or more pharmaceutically acceptable polymer is selected from the group consisting of hydroxypropylmethyl cellulose (HPMC), hydroxypropylmethyl cellulose acetate succinate (HPMCAS) and hydroyxpropylmethyl cellulose phthalate (HPMCP). In particular instances, the one or more pharmaceutically acceptable polymer is HPMC. All other variables are as provided above.

The pharmaceutically acceptable polymer may be selected from vinyl pyrrolidinone/vinyl acetate copolymers. In particular instances, the pharmaceutically acceptable polymer is copovidone, a copolymer of 1-vinyl-2-pyrrolidone and vinyl acetate in the mass proportion of 3:2. Other useful copolymers contain vinyl pyrrolidone and vinyl acetate in ratios of, for example, 90:10, 80:20, 70:30, and 50:50. The amount of vinyl pyrrolidone can range from about 40% up to about 99.9%, and the amount of vinyl acetate can range from about 0.1% up to about 60%. Other vinyl polymers and copolymers having substituents that are hydroxy, alkyl, acyloxy, or cyclic amides include polyethylene polyvinyl alcohol copolymers; and polyvinyl caprolactam-polyvinyl acetate-polyethylene glycol graft copolymer (SoLUPLUs®, BASF Corp.). Commercially available copolymers of vinyl pyrrolidone and vinyl acetate include PLASDONE® S630 (Ashland, Inc., Covonton, Ky.) and KOLLIDON® VA 64 (BASF Corp., Florham Park, N.J.), which contain vinyl pyrrolidone and vinyl acetate in a 60:40 ratio. Other copolymers of vinyl pyrrolidone and vinyl acetate can also be used in the invention. Preferably, the copolymer contains at least 40% vinyl pyrrolidone, although smaller amounts of vinyl pyrrolidone can also be utilized.

In a third aspect of the first embodiment, the one or more pharmaceutically acceptable polymer is present in a concentration of from about 50% w/w to about 95% w/w, based on the total combined weight of the drug substance, polymer, and any optional surfactant (not including other excipients). In particular instances, the one or more pharmaceutically acceptable polymer is present in a concentration of from about 50% w/w to about 90% w/w, or about 70% w/w, based on the total combined weight of the drug substance, polymer, and any optional surfactant (not including other excipients). In this third aspect of the first embodiment, all other variables are as provided above, including the first and second aspects of the first embodiment.

In a fourth aspect of the first embodiment, the action of the polymers may be improved by the presence of a surfactant. Thus, the solid dispersion formulation of the present invention may optionally comprise one or more pharmaceutically acceptable surfactants. The surfactants may increase the rate of dissolution by facilitating wetting, thereby increasing the maximum concentration of dissolved drug. The surfactants may also make the dispersion easier to process. Surfactants may also stabilize the amorphous dispersions by inhibiting crystallization or precipitation of the drug by interacting with the dissolved drug by such mechanisms as complexation, formation of inclusion complexes, formation of micelles, and adsorption to the surface of the solid drug. Surfactants may also facilitate absorption of APIs by altering API permeability and/or efflux directly. See, e.g., Yu et al., 16 PHARM RES. 1812-1817 (1999). Non-limiting examples of pharmaceutically acceptable surfactants that are suitable for the present invention include polyoxyethylene castor oil derivates, e.g. polyoxyethyleneglycerol triricinoleate or polyoxyl 35 castor oil (CREMOPHOR® EL; BASF Corp.) or polyoxyethyleneglycerol oxystearate such as polyethylenglycol 40 hydrogenated castor oil (CREMOPHOR® RH 40, also known as polyoxyl 40 hydrogenated castor oil or macrogolglycerol hydroxystearate) or polyethylenglycol 60 hydrogenated castor oil (CREMOPHOR® RH 60); or a mono fatty acid ester of polyoxyethylene sorbitan, such as a mono fatty acid ester of polyoxyethylene (20) sorbitan, e.g. polyoxyethylene (20) sorbitan monooleate (Tween 80), polyoxyethylene (20) sorbitan monostearate (Tween 60), polyoxyethylene (20) sorbitan monopalmitate (Tween 40), or polyoxyethylene (20) sorbitan monolaurate (Tween 20). Other non-limiting examples of suitable surfactants include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); sucrose fatty acid esters, e.g. sucrose monostearate, sucrose distearate, sucrose monolaurate, sucrose dilaurate; sorbitan fatty acid mono esters such as sorbitan mono laurate (Span 20), sorbitan monooleate, sorbitan monopalnitate (Span 40), or sorbitan stearate; D-α-tocopheryl polyethylene glycol 1000 succinate (vitamin E TPGS); or a combination or mixture thereof. Other non-limiting examples of suitable surfactants include anionic surfactants, e.g. docusate potassium, docusate sodium, docusate calcium and sodium lauryl sulfate (SLS). Other suitable surfactants include, but are not limited to, block copolymers of ethylene oxide and propylene oxide, also known as polyoxyethylene polyoxypropylene block copolymers or polyoxyethylene polypropyleneglycol, such as POLOXAMER® 124, POLOXAMER® 188, POLOXAMER® 237, POLOXAMER® 388, or POLOXAMER® 407 (BASF Corp.). As described above, a mixture of surfactants can be used in a solid composition of the present invention. In specific instances, the surfactant is selected from the group consisting of sodium lauryl sulfate (SLS), D-α-tocopheryl polyethylene glycol 1000 succinate (vitamin E TPGS), or nonionic ethoxylated alcohols like polysorbate or poloxamer. In particular instances, the surfactant is Vitamin E TPGS. In this fourth aspect of the first embodiment, all other variables are as provided above, including the first through third aspects of the first embodiment.

In a fifth aspect of the first embodiment, the one or more pharmaceutically acceptable surfactant is present in a concentration of from about 2% w/w to about 20% w/w, based on the total combined weight of the drug substance, polymer, and surfactant (not including other excipients). In particular instances, the one or more pharmaceutically acceptable surfactant is present in a concentration of from about 5% w/w to about 15% w/w, or about 10% w/w, based on the total combined weight of the drug substance, polymer, and surfactant (not including other excipients). In this fifth embodiment, all other variables are as provided above, including the first through fourth embodiments.

As demonstrated by the Examples, the oral absorption of Compound I when formulated as a solid solution together with one or more pharmaceutically acceptable polymer, such as HPMC, together with one or more surfactants, such as vitamin E TPGS, is superior to formulations based on undispersed amorphous Compound I.

The relative amount of drug, polymer and surfactant can vary widely. The optimal amount of the polymer and surfactant can depend on, for example, the hydrophilic lipophilic balance (HLB), melting point, and water solubility of the copolymer, and the surface tension of aqueous solutions of the surfactant, etc.

The concentration of Compound I can vary from about 0.1% to about 40.0%, from about 5.0% to about 35.0%, or from about 10% to about 30%, by weight based on the total combined weight of the drug substance, polymer, and any optional surfactant (not including other excipients).

The concentration of the pharmaceutically acceptable polymer is added to the concentrations of the Compound I and any optional surfactant to add up to 100%. The concentration can vary from about 0.01% to about 95% by weight based on the total combined weight of the drug substance and polymer, not including other excipients.

An embodiment of the present invention is directed to a solid dispersion formulation that comprises from about 0.1% w/w to about 50% w/w of Compound I or a pharmaceutically acceptable salt thereof, from about 2.0% w/w to about 20% w/w surfactant, with the balance of the formulation being the pharmaceutically acceptable polymer.

In a sixth aspect of the first embodiment, the solid dispersion formulation is in the form of a particle. In instances of the sixth aspect, the particles of the solid dispersion formulation are formed by a process selected from spray drying and extrusion of the composition of the first embodiment. The solid dispersion formulations described herein relate to solid dispersion formulations produced by solvent removal (e.g., spray-drying), introduction of an antisolvent (e.g., precipitation), addition of heat together with mixing (e.g., extrusion), mechanical activation or other means (e.g., to produce a “solid dispersion intermediate”). That is, the solid dispersion formulation may be formed by a process selected from spray drying and extrusion, such as hot-melt extrusion, of the solid dispersion solution. In particular instances, the solid dispersion formulation comprises particles formed by spray drying.

The solid dispersion formulations of the present invention may be prepared by processes that are suitable for causing Compound Ito form a dispersion (also referred to as an amorphous dispersion) in the polymer such that the drug is generally amorphous or dissolved in the polymer or a component of the solid dispersion formulation, such as a surfactant. The dispersions are stable, and the drug does not form crystals or other insoluble particles. Such methods include solution methods, such as spray drying, spray coating, freeze-drying, and evaporation of a co-solvent under vacuum or by heating a solution of polymer and drug. Such methods also include methods that blend the solid drug with the polymer in the molten state, such as hot-melt extrusion, and methods of compounding the solid non-molten polymer and drug under heat and pressure to form a dispersion. If the dispersion is effectively a homogeneous molecular dispersion of the individual components, it may also be described as a solid solution, a specific subclass of solid dispersions.

Spray drying is well known (see, e.g., Masters, Spray Drying Handbook, 1991, 5^{th} edition, Longman Scientific & Technical) and widely practiced in a variety of industrial applications including spray drying of milk (see, e.g., U .S. Pat. No. 4,187,617) and pharmaceutical products (see, e.g., U .S. Pat. No. 6,763,607). In spray drying, the polymer, API, and optionally a surfactant, are dissolved in a solvent and then are sprayed through a nozzle as a fine spray into a chamber where the solvent is evaporated quickly to make particles comprising polymer, API, and surfactant. Ideally, the solvent is any solvent in which all of the components of the solid dispersion formulation are soluble and that is readily evaporated in a spray dryer. The solvent should also be suitable for use in preparing pharmaceutical compositions. In certain embodiments of the invention, the use of mixed-solvent systems, particularly those containing a combination of water and another solvent, are necessary to facilitate the production of solid solution intermediates containing Compound I, an absorption enhancing polymer or polymer(s), and, optionally a surfactant.

Useful solvents for spray drying include water, acetone, ethanol, methanol, dichloromethane, isopropanol and tetrahydrofuran (THF). The spray drying may be performed in a mixed-solvent system. A mixed-solvent system is a solvent system that comprises a first solvent and a second solvent. In aspects, the first solvent may be selected from the group consisting of acetone, ethanol, methanol, dichloromethane, isopropanol and tetrahydrofuran (THF); the second solvent is water. In specific instances, the first solvent is acetone and the second solvent is water. In particular aspects, the first solvent may be selected from the group consisting of ethanol, methanol and acetone; the second solvent is water. In specific instances, the first solvent is acetone and the second solvent is water. The proportions of the first solvent to second solvent may be about 90:10, about 80:20, about 70:30, or about 60:40. Mixed-solvent systems are described in International Patent Application Publication No. WO2007/109605 and U.S. Patent Application Publication No. US2007/0026083. Solids loading, which usually refers to the concentration of solid components in the spray-drying solvent system, does not typically exceed 50% and depends on solution properties, such as solubility, stability and viscosity. The solids, comprising Compound I, the pharmaceutically acceptable polymer and surfactant, are present in the spray drying solution in a concentration of from about 1% w/w to about 50% w/w, based on the solubility, stability and viscosity of the solution. In particular instances, the solids are present in the solution in a concentration of from about 5% w/w to about 25% w/w.

Following formation of a solid dispersion, the resulting spray-dried intermediate can undergo a secondary drying step to remove residual solvents. This secondary drying unit operation can occur in a static dryer or agitated dryer. Gas, humidified gas, or vacuum may be applied to the material in the secondary dryer and such application can be useful in more rapidly removing residual solvents that remain in the spray-dried intermediate. See, e.g., European Patent Application No. EP1855652 A2 (and references therein) and International Patent Application Publication No. WO2008012617A1 (and references therein).

In hot-melt extrusion, the polymer, drug, and any optional surfactant may be either premixed together (e.g., via a wet granulation process) or fed as independent feed streams into the extruder (see Polymer Extrusion 4^{th} Edition by Chris Rauwendaal 2001, Hanser Gardner Publications, Inc., Cincinnati, OH or Schenck et al., (2010), Achieving a Hot Melt Extrusion Design Space for the Production of Solid Solutions, in Chemical Engineering in the Pharmaceutical Industry: R&D to Manufacturing (ed. D. J. am Ende), John Wiley & Sons, Inc., Hoboken, N.J., USA). In accordance with this embodiment, any means for preparing a melt in any convenient apparatus in which an admixture of Compound I, a pharmaceutically acceptable polymer and optionally a surfactant can be heated and optionally mixed can be used. Solidification can be carried out by merely cooling the melt. Once a solid is obtained, the solid can be further mechanically processed to provide a convenient form for incorporation into a medicament, for example, tablets or capsules.

It will be appreciated that other methods of preparing a melt, solidifying it, and forming the solid into conveniently sized particles can be utilized without departing from the spirit of the invention. For example, compositions of the invention may be prepared using an extruder. When an extruder is employed to prepare compositions of the invention, the material may be introduced into the extruder either in a pre-flux state, that is, as a dry admixture, or in a fluxed state, that is in a melted, plastic, or semi-solid state achieved after the application of sufficient heat to the admixture to cause Compound Ito dissolve in the polymer, optionally when a fluxed charge is prepared, blending may be employed during heating to promote uniformity of the fluxed material.

If the material is introduced to the extruder in a fluxed state, residence time in the extruder is selected to be just sufficient to ensure homogeneity of the composition and the temperature is preferably maintained in the extruder at a level just sufficient to insure that the material maintains its plasticity so that it can be extruded into a conveniently shaped extrudate. If the material is introduced into an extruder in a pre-flux state, the extruder components, for example, the barrels and any mixing chamber present in the equipment, will be maintained at a temperature sufficient to promote fluxing of the admixture. Temperatures selected for use in processing a composition will also take into account that blending which occurs within the extruder equipment, for example, in a mixing section of the barrels, will also contribute to localized fluxing of the admixture by imparting shear-stresses that induce heating in the mixture. Additionally it will be appreciated that equipment temperatures and residence times will be selected to minimize the amount of time that the admixture placed into the extruder spends under conditions of heating and/or shear stress so as to minimize the amount of Compound I, which is decomposed during formation of the composition, as discussed above. In general, extrusion processes in which heating is applied to the material extruded are termed “hot-melt/extrusion processes.” When compositions of the present invention are prepared using extrusion equipment, the extrudate thus provided can be in any convenient shape, for example, noodles, cylinders, bars, or the like. If desired, the extrudate can be further processed, for example by milling, to provide a particulate form of the composition.

### Blended Compositions

A second embodiment relates to blended compositions that comprise the solid dispersion formulation as described in the above embodiment, one or more of a diluent, disintegrant, salt, lubricant and glidant. In all aspects of this embodiment, all variables are as provided in the first embodiment and all aspects thereof

In a first aspect of the second embodiment, the solid dispersion formulation is present in the blended composition in a concentration of from about 20% w/w to about 60% w/w. In particular instances, the solid dispersion formulation is present in the blended composition in a concentration of from about 30% w/w to about 50% w/w, or about 45% w/w.

In a second aspect of the second embodiment, a diluent in the blended composition is one or more pharmaceutically acceptable diluents selected from the group consisting of mannitol, microcrystalline cellulose, calcium carbonate, sodium carbonate, lactose, calcium phosphate, sodium phosphate and kaolin, and combinations thereof In particular instances, the diluent is one or more selected from the group consisting of lactose, microcrystalline cellulose, mannitol and dicalcium phosphate. In a particular instance, the diluent is lactose and microcrystalline cellulose.

In a third aspect of the second embodiment, the diluent is present in the blended composition in a concentration of from about 10% w/w to about 40% w/w. In particular instances, the diluent is present in the blended material in a concentration of from about 15% w/w to about 35% w/w, or about 25% w/w.

In a fourth aspect of this second embodiment, a disintegrent is selected from the group consisting of croscarmellose sodium, sodium starch glycolate and crospovidone. In particular instances, the disintegrant is croscarmellose sodium.

In a fifth aspect of the second embodiment, the disintegrant is present in the blended composition in a concentration of from about 5% w/w to about 25% w/w. In particular instances, the disintegrant is present in a concentration of from about 10% w/w to about 20% w/w, or about 15% w/w.

In a sixth aspect of the second embodiment, a salt is selected from the group consisting of NaCl, KCl, CaCl₂, KH₂PO₄, NaH₂PO₄, K₂SO₄, NaHCO₃, K₂CO₃ and combinations thereof. In aspects, the salt in the blended composition is selected from the group consisting of NaCl, KCl, CaCl₂ and combinations thereof In a particular instance, the salt is NaCl.

In a seventh aspect of the second embodiment, the salt is present in the blended composition in a concentration of from about 2% w/w to about 20% w/w. In particular instances, the salt is present in a concentration of from about 5% w/w to about 15% w/w, or about 10% w/w.

In an eighth aspect of this second embodiment, a lubricant is selected from the group consisting of magnesium stearate and sodium starch fumarate. In a particular instance, the lubricant is magnesium stearate.

In a ninth aspect of the second embodiment, the lubricant is present in the blended composition in a concentration of from about 0.25% w/w to about 5% w/w. In particular instances, the lubricant is present in a concentration of from about 0.5% w/w to about 2% w/w, or about 1% w/w.

In a tenth aspect of this second embodiment, a glidant is selected from the group consisting of starch, talc, magnesium stearate and silicon dioxide and combinations thereof In a particular instance, the glidant is silicon dioxide.

In an eleventh aspect of the second embodiment, the glidant is present in the blended composition in a concentration of from about 0.1% w/w to about 1% w/w. In particular instances, the glidant is present in a concentration of from about 0.2% w/w to about 0.8% w/w, or about 0.5% w/w.

In any single aspect of the second embodiment, all other aspects and variables are as described in the first and second embodiments.

### Oral Dosage Forms

In a third embodiment, the blended composition is formulated as an oral dosage form. In aspects of this embodiment, the oral dosage form is a tablet or as a capsule.

A fourth embodiment of the invention is directed to a process for preparing a solid pharmaceutical composition comprising the steps of: a) dissolving a solid dispersion formulation as described herein in a mixed-solvent system; b) spray drying the dissolved formulation to form particles; and c) compressing the particles into a tablet or filling into a capsule. In addition, the tablet is optionally film-coated; in further aspects, the tablet or capsule is optionally photo-shielded, for example by use of a blister packaging.

In particular aspects of the process according to the fourth embodiment, the process comprises the steps of: a) dissolving a solid dispersion formulation as described herein in a mixed-solvent system; b) spray drying the dissolved formulation to form particles; c) blending the particles with one or more of a diluent, disintegrant, salt, lubricant and glidant; d) subjecting the blend of step c) to roller compaction; e) adding a lubricant; and f) compressing the particles into a tablet or filling into a capsule. In addition, the tablet is optionally film-coated; in further aspects, the tablet or capsule is optionally photo-shielded, for example by use of a blister packaging.

In aspects of the fourth embodiment, the diluents, disintegrents, salts, lubricants and/or glidants are as described above with respect to blended compositions. The diluents, disintegrents, salts, lubricants and/or glidants may be present in the concentrations described above with respect to blended compositions.

Pharmaceutical dosage forms intended for oral use may be prepared from the solid dispersions described above in accordance with the methods described herein and other methods known to the art for the manufacture of pharmaceutical compositions. Such compositions may further contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as mannitol, microcrystalline cellulose, calcium carbonate, sodium carbonate, lactose, calcium phosphate or sodium phosphate; granulating and disintegrating agents, for example, croscarmellose sodium, sodium chloride, corn starch, or alginic acid; binding agents, for example starch, gelatin or acacia; glidants such as colloidal silicon dioxide, lubricating agents, for example magnesium stearate, sodium stearyl fumarate, stearic acid or talc, and antioxidants, for example, propyl gallate, butylated hydroxyanisole and butylated hydroxy toluene. The tablets may be uncoated or they may be coated to delay disintegration and absorption in the gastrointestinal tract and thereby provide a sustained action over a longer period. Compositions for oral use may also be presented as capsules (e.g., hard gelatin) wherein the active ingredient is mixed with an inert solid diluent, for example, calcium carbonate, calcium phosphate or kaolin, or as soft gelatin capsules wherein the active ingredient is mixed with liquids or semisolids, for example, peanut oil, liquid paraffin, fractionated glycerides, surfactants or olive oil. Aqueous suspensions contain the active materials in admixture with excipients suitable for the manufacture of aqueous suspensions. Dispersible powders and granules suitable for preparation of an aqueous suspension by the addition of water provide the active ingredient in admixture with a dispersing or wetting agent, suspending agent and one or more preservatives. In certain embodiments of the invention, the pharmaceutical dosage forms of the invention include a diluent system, lubricant and glidant, at concentrations of from about 0% w/w to about 70% w/w, from about 0.25% w/w to about 3.0% w/w, from about 0% w/w to about 3.0% w/w, respectively, or from about 20% w/w to about 60% w/w, from about 0.5% w/w to about 2.0%, and from about 0% w/w to about 1.0% w/w, respectively. In certain embodiments of the invention, the solid dispersion formulations is blended with a diluent, one or more disintegrating agents, lubricant and glidant. An exemplary formulation includes mannitol, croscarmellose sodium, sodium chloride, colloidal silica and magnesium stearate.

The blended compositions may be roller compacted or wet granulated to densify and/or reduce the risk of segregation of components during subsequent handling (e.g., compression into tablets). Granulation steps can also be used to minimize the impact of raw material property variability (e.g., excipient particle size) on subsequent processing (e.g., tablet compression) and ultimate product performance. Lubrication is typically performed prior to roller compaction and tablet compression to reduce the tendency of material to adhere to compression surfaces (e.g., tablet tooling). A preferred lubricant is magnesium stearate. These methods can be carried out by those skilled in the art. See, e.g., Ansel, Introduction to Pharmaceutical Dosage Forms, Seventh Edition, 1999.

To prepare the pharmaceutical dosage form of the invention, the solid dispersion is compressed into an oral dosage form including tablets or capsules. Tablets can be prepared with a variety of possible shapes (ellipsoidal, capsule, biconvex round, etc.). The powder can also be encapsulated in capsule dosage (e.g., using hard gelatin capsules). Techniques suitable for preparing solid oral dosage forms of the present invention are described in Remington's Pharmaceutical Sciences, 18th edition, edited by A. R. Gennaro, 1990, Chapter 89 and in Remington - The Science and Practice of Pharmacy, 21st edition, 2005, Chapter 45. In certain embodiments, the solid dispersion formulation is present in an amount of from about 5% w/w to about 75% w/w of the pharmaceutical dosage form or from about 30% w/w to about 50% w/w of the final pharmaceutical dosage form.

### Fixed-Dose Combination Dosage Forms

Additional embodiments include combination regimens, including fixed-dose combinations. For combination regimens, other drug substance(s) can be added to the solid solution or the tablet formulation, either in a crystalline form, neat amorphous form, or as a solid solution. In particular combination regimens, an additional drug substance is formulated into a solid dispersion formulation, and the solid dispersion formulation of Compound I and the solid dispersion formulation of the additional drug substance are combined into a blended composition and provided as a dosage form that may be a tablet or capsule. Additional components may also be combined into the blended composition, such as diluents, disintegrants, salts, lubricants and glidants, as described above.

Exemplary drug substances include, but are not limited to, HCV protease inhibitors, HCV polymerase inhibitors, HCV NS4A inhibitors and HCV NS5A inhibitors.

HCV protease inhibitors include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Application Publication Nos. US2002/0068702, US2002/0160962, US2005/0119168, US2005/0176648, US2005/0209164, US2005/0249702 and US2007/0042968; and International Patent Application Publication Nos. WO03/006490, WO03/087092, WO04/092161 and W08/124148.

HCV protease inhibitors also include, but are not limited to, SCH503034 (Boceprevir, Schering-Plough), SCH900518 (Schering-Plough), VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhbitors include, but are not limited to, those disclosed in Landro et al., 36(31) BIOCHEMISTRY 9340-9348 (1997); Ingallinella et al., 37(25) BIOCHEMISTRY 8906-8914 (1998); Llinds-Brunet et al., 8(13) BIOORG. MED. CHEM. LETT. 1713-1718 (1998); Martin et al., 37(33) BIOCHEMISTRY 11459-11468 (1998); Dimasi et al., 71(10) J. VIROL. 7461-7469 (1997); Martin et al., 10(5) PROTEIN ENG. 607-614 (1997); Elzouki et al., 27(1) J. HEPAT. 42-48 (1997); 9(217) BIOWORLD TODAY 4 (November 10, 1998); U.S. Patent Application Publication Nos. US2005/0249702 and US 2007/0274951; and International Patent Application Publication Nos. WO98/14181, W098/17679, WO98/17679, WO98/22496, WO99/07734 and WO05/087731.

HCV polymerase inhibitors include, but are not limited to, VP-19744 (Wyeth/ViroPharma), PSI-7851 (Pharmasset), GS-7977 (sofosbuvir, Gilead), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK- 3281 (Merck), VCH222 (ViroChem), VCH916 (ViroChem), VCH716(ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., 7(4) CURRENT OPINION IN DRUG DISCOVERY AND DEVELOPMENT 446 (2004); Tan et al., 1 NATURE REVIEWS 867 (2002); and Beaulieu et al., 5 CURRENT OPINION IN INVESTIGATIONAL DRUGS 838 (2004).

HCV NS4A inhibitors include, but are not limited to, those disclosed in U.S. Pat. Nos. 7,476,686 and 7,273,885; U.S. Patent Application Publication No. US2009/0022688; and International Patent Application Publication Nos. WO2006/019831 and WO2006/019832. Additional HCV NS4A inhibitors include, but are not limited to, AZD2836 (Astra Zeneca) and ACH-806 (Achillon Pharmaceuticals, New Haven, Conn.).

A further embodiment of the invention is directed to a process for preparing a solid pharmaceutical composition comprising the steps of: a) dissolving a solid dispersion formulation comprising Compound I as described herein and a solid dispersion formulation comprising a second drug substance in a mixed-solvent system; b) spray drying the solution to form particles comprising Compound I and the second drug substance to form particles; and c) compressing the particles into a tablet or filling into a capsule. In addition, the tablet is optionally film-coated; in further aspects, the tablet or capsule is optionally photo-shielded, for example by use of a blister packaging.

In particular aspects of the process according to this embodiment, the process comprises the steps of: a) dissolving a solid dispersion formulation comprising Compound I as described herein and a solid dispersion formulation comprising a second drug substance in a mixed-solvent system; b) spray drying the solution to form particles; c) blending the particles with one or more of a diluent, disintegrant, salt, lubricant and glidant; d) subjecting the blend of step c) to roller compaction; e) adding a lubricant; and f) compressing the particles into a tablet or filling into a capsule. In addition, the tablet is optionally film-coated; in further aspects, the tablet or capsule is optionally photo-shielded, for example by use of a blister packaging.

In a first aspect of this embodiment, the solid dispersion formulation, diluent, disintegrant, salt, lubricant and glidant are as described above.

The following examples serve only to illustrate the invention and its practice. The examples are not to be construed as limitations on the scope or spirit of the invention.

In addition, the following abbreviations are used throughout this specification and in the Examples. Each of these terms has the meaning listed below.

### ABBREVIATIONS

CI Confidence interval
GM Geometric mean
AUC_{0-∞} Area under the concentration time curve from time zero to infinity
GMR Geometric mean ratio
HPMC Hydroxypropylmethyl cellulose
TPGS Vitamin E polyethylene glycol succinate
psi Pounds per square inch [gauge], 1 Pascal=0.000145037738007 psi
% w/w Percentage by weight (i.e., the number of grams of solute in 100g of solution)
kg Kilogram(s)
g Gram(s)
hr Hour(s)
min Minute(s)
Cₘₐₓ Maximum concentration (specifically of a drug)
nM Nanomolar
μM Micromolar

### EXAMPLES

### Example 1:

### Conventional Roller Compacted Formulation 1

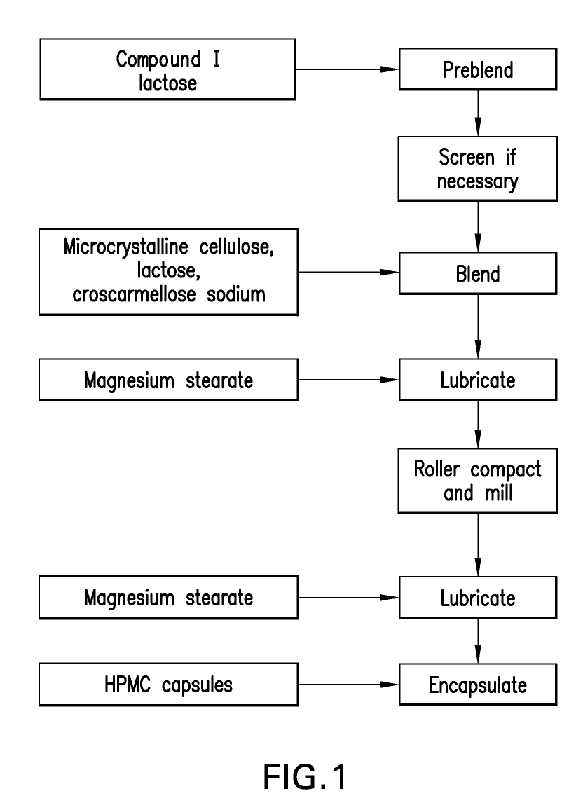

The pharmacokinetics of a conventional roller compacted formulation of Compound I was examined. As described below, an amorphous form of Compound 1, as described in U.S. Pat. No. 8,871,759, in particular Example 223, was roller compacted and filled into capsules to produce the formulation with a composition outlined in Table 1 below. FIG. 1 outlines the process used to make Formulation 1.

TABLE 1
Composition of Formulation 1

| Components | mg/tablet |
|---|---|
| Compound I | 100.0 |
| Microcrystalline cellulose | 199.6 |
| Lactose monohydrate | 199.6 |
| Croscarmellose sodium | 15.60 |
| Magnesium Stearate | 5.200 |
| Total | 520.0 |

The oral absorption obtained from Formulation 1 with and without pH-modifying medications was evaluated as part of human clinical study. Formulation 1 was dosed by itself (Treatment 1) and in the presence of a medication that raises gastric pH (Treatment 2). The results are shown in Table 2.

TABLE 2
Summary of Human PK Results from Biocomparison
Study (100 mg dose; Healthy Subjects)

| | | Treatment 2 | |
|---|---|---|---|
| | | Compound 1 | |
| | Treatment 1 | with pH-raising | Treatment 2/ |
| | Compound I | medication | Treatment 1 |
| | [GM and 95% CI] | [GM and 95% CI] | [GMR and 90% CI] |
| AUC_{0-∞} | 3.77 | 1.57 | 0.42 |
| (μM · hr) | [2.90, 4.90] | [1.25, 1.99] | [0.33, 0.53] |
| Cₘₐₓ | 216 | 76.2 | 0.35 |
| (nM) | [150, 310] | [54.6, 106] | [0.27, 0.47] |

When co-dosed with a medication that raises gastric pH, AUC_{0-∞} of Formulation 1 decreases by 47-67%, and Cₘₐₓ decreases by 53-73%. This example illustrates the impact on absorption, relative to using a conventional formulation of Compound I, for a solid solution based formulation upon co-administration with a pH-raising medication.

### Example 2:

### HPMC-TPGS Formulation of Compound I

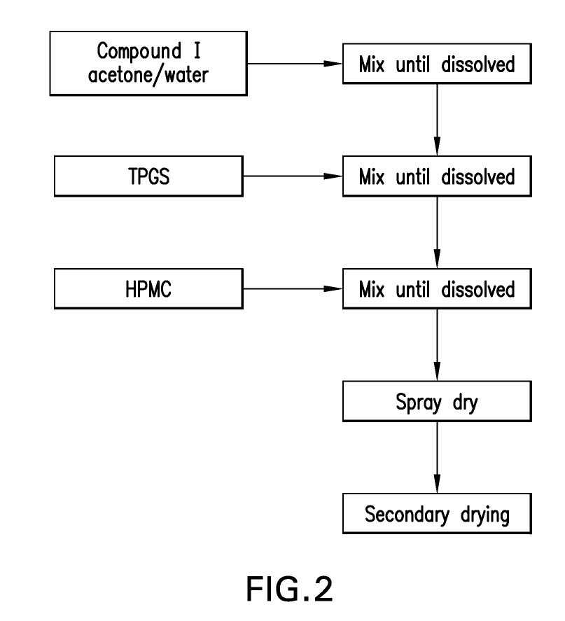

Formulation 2 is a tablet composition containing a solid dispersion of Compound I as shown in Table 3. The solid dispersion was prepared from a solution comprising Compound I, TPGS, and HPMC by spray drying from an acetone/water solvent system, as shown in FIG. 2.

TABLE 3
Composition of the Tablet Formulation 2

| Components | mg/tablet |
|---|---|
| Compound I | 50.00 |
| Hypromellose 2910 (HPMC) | 175.0 |
| Vitamin E Polyethylene Glycol Succinate (TPGS) | 25.00 |

A NIRO PSD-2 spray dryer with a pressure nozzle was used to produce the spray-dried particles. The spray-dried particles are dried in a chamber that can contain an inert heated gas (e.g., nitrogen). The particles thus produced are collected (e.g., using a cyclone). Typically, a secondary drying operation is used to sufficiently dry the spray-dried intermediate. Humid nitrogen or air may be used to facilitate drying. Tray dryers or agitated dryers can be used to perform this secondary drying operation.

Heated nitrogen was supplied to the spray dryer at an inlet temperature sufficient to maintain a 50° C. outlet temperature and a gas flow rate of 1856 g/min. The spray drying solution flow rate was 7.14 Kg/hr that required a nozzle pressure of approximately 221 psi using a STEINEN A75 nozzle.

### Example 3:

### Tablet Composition HPMC-TPGS Formulation of Compound I

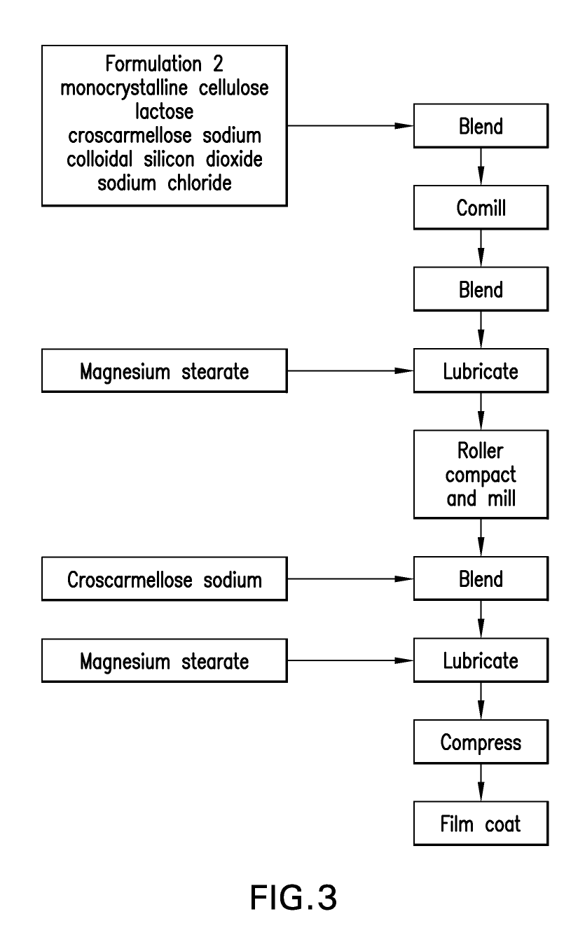

A tablet composition, Formulation 3, was prepared with a composition described in Table 4 and as shown in FIG. 3. The Formulation 2 spray dried intermediate was blended and roller compacted along with microcrystalline cellulose, lactose, croscarmellose sodium, sodium chloride, colloidal silicon dioxide, and magnesium stearate. Only half of the magnesium stearate was added prior to roller compaction. The second half of the magnesium stearate and the remaining croscarmellose sodium was added after roller compaction and the resulting powder mix was blended further. A rotary tablet press was used to produce tablets of 50 mg potency.

TABLE 4
Composition of Formulation 3

| Components | mg/tablet |
|---|---|
| Formulation 2 | 250 |
| Microcrystalline cellulose | 71.25 |
| Lactose | 71.25 |
| Croscarmellose sodium | 82 |
| Sodium chloride | 50 |
| Colloidal silicon dioxide | 2.5 |
| Magnesium stearate | 5.0 |
| Film coat | 16 |
| Total tablet weight | 548.0 |

### Example 4:

### Co-Administration with a Protease Inhibitor

The oral absorption obtained from grazoprevir and Formulation 3 with (Treatment 2) and without (Treatment 1) co-administration of a pH-raising medication was evaluated as part of human clinical study. The results are shown in Table 5.

TABLE 5
Comparison of Human pK Results from Biocomparison Study
(100 mg grazoprevir/50 mg Compound I; Healthy Subjects)

| | | | | | Treatment 2/ |
|---|---|---|---|---|---|
| | Treatment 2 | | Treatment 1 | | Treatment 1 |
| | | GM | | GM | GMR |
| Parameters | N | [95% CI] | N | [95% CI] | [90% CI] |
| AUC0-∞ | 12 | 1793.117 | 12 | 1973.325 | 0.91 |
| (nM · hr) | | [1398, 2299] | | [1557, 2501] | [0.74, 1.12] |
| Cmax | 12 | 95.535 | 12 | 102.481 | 0.93 |
| (nM) | | [74.4, 123] | | [83.7, 125] | [0.74, 1.17] |

The exposure of Compound I following co-administration of Formulation 3 and a pH-raising medication is comparable to that when Formulation 3 is administered alone.

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A solid dispersion formulation comprising:
(a) dimethyl N,N′-([(6S)-6-phenylindolo[1,2-c] [1,3 ]1) enzoxazine-3,10-diyl[bis {1H-imidazole-5,2-diyl-(2S)-pyrrolidine-2,1-diyl [(2S)-3-methyl-1-oxobutane-1,2-diyl]})dicarbamate (Compound I): 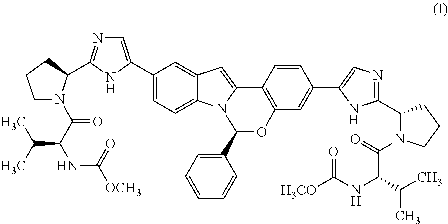 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]
or a pharmaceutically acceptable salt thereof;
(b) one or more pharmaceutically acceptable polymers or a mixture thereof; and
(c) optionally one or more pharmaceutically acceptable surfactants or a mixture thereof;
wherein Compound I and the one or more surfactants are dispersed in a polymer matrix formed by the one or more pharmaceutically acceptable polymers; and
wherein Compound I is substantially amorphous.

2. (canceled)

3. The solid dispersion formulation according to claim 1, wherein Compound I, or a pharmaceutically acceptable salt thereof, is present in a concentration of about 5% w/w to about 50% w/w.

4. The solid dispersion formulation according to claim 3, wherein Compound I, or a pharmaceutically acceptable salt thereof, is present in a concentration of about 10% w/w to about 40% w/w.

5. The solid dispersion formulation according to claim 4, wherein Compound I, or a pharmaceutically acceptable salt thereof, is present in a concentration of about 20% w/w.

6. The solid dispersion formulation according to claim 1, wherein the pharmaceutically acceptable polymer is selected from the group consisting of cellulosic polymers.

7. The solid dispersion formulation according to claim 6, wherein the pharmaceutically acceptable polymer is hydroxypropylmethyl cellulose.

8. The solid dispersion formulation according to claim 1, wherein the pharmaceutically acceptable polymer is present in a concentration of about 50% w/w to about 95% w/w.

9. The solid dispersion formulation according to claim 8, wherein the pharmaceutically acceptable polymer is present in a concentration of about 60% w/w to about 90% w/w.

10. The solid dispersion formulation according to claim 9, wherein the pharmaceutically acceptable polymer is present in a concentration of about 70% w/w.

11. The solid dispersion formulation according to claim 1, wherein the pharmaceutically acceptable surfactant is present and is selected from the group consisting of sodium lauryl sulfate, D-α-tocopheryl polyethylene glycol 1000 succinate, a polysorbate and a poloxamer.

12. The solid dispersion formulation according to claim 11, wherein the pharmaceutically acceptable surfactant is D-α-tocopheryl polyethylene glycol 1000 succinate.

13. The solid dispersion formulation according to claim 1, wherein the pharmaceutically acceptable surfactant is present in a concentration of about 2% w/w to about 20% w/w.

14. The solid dispersion formulation according to claim 13, wherein the pharmaceutically acceptable surfactant is present in a concentration of about 5% w/w to about 15% w/w.

15. The solid dispersion formulation according to claim 14, wherein the pharmaceutically acceptable surfactant is present in a concentration of about 1% w/w.

16. The solid dispersion formulation according to claim 1, wherein the solid dispersion formulation comprises particles.

17. The solid dispersion formulation according to claim 16, wherein said solid dispersion formulation comprises particles formed by spray-drying or extruding.

18. The solid dispersion formulation according to claim 17, wherein said solid dispersion formulation comprises particles wherein the surfactant is D-α-tocopheryl polyethylene glycol 1000 succinate and is formed by spray drying in a mixed-solvent system.

19. The solid dispersion formulation according to claim 18, wherein the mixed-solvent system comprises at least a first solvent and at least a second solvent, wherein said first solvent is selected from the group consisting of acetone, ethanol, methanol, dichloromethane, isopropanol and tetrahydrofuran (THF), and mixtures thereof, and said second solvent is water.

20. The solid dispersion formulation of claim 19, wherein in the mixed-solvent system, the first solvent is acetone and the second solvent is water.

21. A blended composition comprising the solid dispersion formulation according to claim 16, and one or more of a diluent, disintegrant, salt, lubricant and glidant.

22. The blended composition according to claim 21, wherein the diluent is selected from the group consisting of mannitol, microcrystalline cellulose, calcium carbonate, sodium carbonate, lactose, calcium phosphate, sodium phosphate and kaolin and combinations thereof.

23. The blended composition according to claim 21, wherein the disintegrant is selected from the group consisting of croscarmellose sodium, sodium starch glycolate and crospovidone and combinations thereof.

24. The blended composition according to claim 21, wherein the salt is selected from the group consisting of NaCl, KCl, and CaCl₂ and combinations thereof.

25. The blended composition according to claim 21, wherein the lubricant is selected from the group consisting of magnesium stearate and sodium starch fumarate and combinations thereof.

26. The blended composition according to claim 21, wherein the glidant is selected from the group consisting of starch, talc, magnesium stearate and silicon dioxide and combinations thereof.

27. The blended composition according to claim 21, wherein the solid dispersion formulation is present at a concentration of from about 20% w/w to about 60% w/w of the composition.

28. An oral dosage form comprising the blended composition according to claim 21, wherein the blended composition is formulated as a tablet or as a capsule.

29. A process for preparing an oral dosage form comprising the steps of:
a) dissolving the solid dispersion formulation in a mixed-solvent system, wherein the soild dispersion formulation comprises (a) dimethyl N,N′-([6S)-6-phenylindolo[1,2-c][1,3]benzoxazine-3,10-diyl[bis{1H-imidazole-5,2-diyl-(2S)-pyrrolidine-2,1-diyl[(2S)-3-methyl-1-oxobutane-1,2-diyl[}dicarbamate (Compound I): 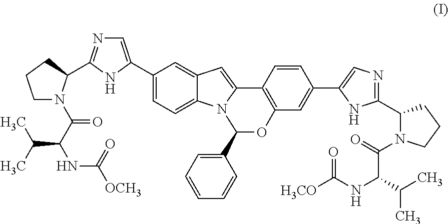 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]
or a pharmaceutically acceptable salt thereof, (b) one or more pharmaceutically acceptable polymers or a mixture thereof, and (c) optionally one or more pharmaceutically acceptable surfactants or a mixture thereof, and wherein Compound I and the one or more surfactants are dispersed in a polymer matrix formed by the one or more pharmaceutically acceptable polymers, and v/herein Compound I is substantially amorphous;
b) spray drying the dissolved solid dispersion formulation to form particles; and
c) compressing the particles into a tablet or filling into a capsule.

30. The process according to claim 29, comprising the steps of:
a) dissolving the solid dispersion formulation in mixed-solvent system;
b) spray drying the dissolved solid dispersion formulation to form particles;
c) blending the particles with one or more of a diluent, disintegrant, salt, lubricant and glidant;
d) subjecting the blend of step c) to roller compaction;
e) adding a lubricant; and
f) compressing the particles into a tablet or filling into a capsule.

31. The process according to claim 29, wherein the mixed-solvent system comprises at least a first solvent and at least a second solvent, wherein said first solvent is selected from the group consisting of acetone, ethanol, methanol, dichloromethane, isopropanol and tetrahydrofuran (THF), and mixtures thereof, and said second solvent is water.

32. The process according to claim 31, wherein in the mixed-solvent system, the first solvent is acetone and the second solvent is water.
